(19) United States
(12) Patent Application Publication
(10) Pub. No.: US 2024/0057967 A1
(43) Pub. Date: Feb. 22, 2024

(54) TRANSCRANIAL ULTRASOUND DEVICES AND METHODS

(71) Applicant: UCL BUSNIESS LTD, London Greater London (GB)

(72) Inventors: Bradley TREEBY, London Greater London (GB); Olivia WRIGHT, London Greater London (US); Eleanor MARTIN, London Greater London (US)

(21) Appl. No.: 18/260,964

(22) PCT Filed: Jan. 14, 2022
(86) PCT No.: PCT/IB2022/050293

§ 371 Date: Jul. 11, 2023

(30) Foreign Application Priority Data
Jan. 14, 2021 (GB) 2100450.2

(51) Int. Cl. A61B 8/08; A61B 8/00; A61N 7/00

(52) U.S. Cl. CPC A61B 8/0808; A61B 8/4494; A61N 7/00; A61N 2007/0078

(57) ABSTRACT

A transcranial ultrasound cap (100) is provided. The cap (100) comprises: a bowl (102) having an inner volume arranged to receive a portion of a human head (200), the bowl (102) conforming to the shape of an ellipsoid; and a transducer array (110) in the inner volume, comprising a plurality of ultrasound transducers (112) coupled to the bowl (102).

### FIELD OF INVENTION

The present disclosure relates generally to devices and methods for transcranial ultrasound.

### BACKGROUND

The structures in the center of the brain (often referred to as the deep grey matter structures) are vitally important to our ability to perform everyday tasks. This includes processing and passing on information from our senses, regulating consciousness and sleep, and the control of voluntary movement and coordination. Abnormalities in the deep grey matter structures can lead to a wide range of neurological disorders. Some examples are Parkinson's disease, Huntington's disease, chronic pain, and essential tremor. These disorders are extremely debilitating and have a significant impact on quality of life. Neurological conditions are very common and currently form the largest single cause of morbidity in the EU in terms of disability adjusted life years.

Currently, most neurological disorders are treated by the prescription of drugs that cause alterations in brain function. These drugs act on the structures that transmit electrical and chemical signals in the brain. For many patients, this causes a reduction in their symptoms. However, long-term treatment is often not very effective, and there can be many side-effects. For some patients with advanced or drug-resistant disorders, a surgical procedure known as deep brain stimulation may also be offered. This involves putting a small wire into the brain via holes drilled through the skull. This can be very effective, but is highly invasive, and only available to a small number of patients.

It is also possible to modulate brain activity by applying or inducing an electric current through the skull using non-invasive brain stimulation. In particular, transcranial magnetic stimulation (TMS) and transcranial direct current stimulation (tDCS) have both been used for stroke rehabilitation, are FDA approved for treatment of migraine and depression, and are currently in clinical trials for a wide range of other neurological and psychiatric conditions, including pain and epilepsy. However, these techniques are limited to superficial layers of the brain due to the rapid decay of the electromagnetic field with distance, so cannot be used for disorders associated with the deep brain. Moreover, they are non-specific, with a spatial resolution on the order of centimetres across the cortical surface.

Non-invasive brain stimulation has also been demonstrated using low-intensity ultrasound waves, a technique called transcranial ultrasound stimulation or TUS (other acronyms are also used). In this technique, focused ultrasound waves are used to alter the gating dynamics of neuronal ion channels. Depending on the pattern of the ultrasound pulses, this can cause the generation or suppression of electrical signals in the brain, which in turn can potentially be used to restore normal brain function. More than 16 studies (at the time of filing) in humans have been reported in the journal literature, with many more reported in animal models. These studies show that TUS is both safe and effective at modulating brain activity.

Ultrasound stimulation has several key advantages compared to existing techniques: (1) it is completely non-invasive, (2) the ultrasound waves can be easily transmitted through the skull and into the deep brain, (3) the ultrasound can be focused to a diffraction limited focal spot size, giving high targeting specificity on the order of millimetres. Although studies to date have largely been limited to the stimulation of superficial brain regions, these advantages open up the possibility of ultrasound being the first non-invasive modality for deep brain stimulation.

There are several other ultrasound therapies that use focused ultrasound in the brain. In MR-guided focused ultrasound surgery (also called high-intensity focused ultrasound), high-intensity ultrasound is focused to a small spot and used to heat up and destroy (ablate) brain tissue. This technique has been used to ablate tissue in the deep brain to treat essential tremor, tremor dominant Parkinson's disease, and neuropathic pain. In ultrasound-induced blood-brain barrier disruption, focused ultrasound is used in conjunction with injected microbubbles to increase the permeability of the blood-brain barrier which allows focal delivery of drugs into the brain.

In addition to ultrasound therapy, arrays of ultrasound transducers positioned around the head may also be used for imaging. For example, in ultrasound computed tomography, an array of ultrasound transducers is used to transmit and receive ultrasound pulses. The recorded signals are then used to reconstruct images of the acoustic properties of the skull and brain. Similarly, in photoacoustic tomography, an array of ultrasound transducers is used to detect ultrasound waves generated using pulsed laser light. The recorded signals are then used to reconstruct images of optical absorption, for example, related to hemoglobin concentration within the brain.

There is a want in the field for improved non-invasive methods to stimulate the brain, or otherwise perform therapeutic and diagnostic ultrasound applications in the brain, in particular methods that reach the deep brain. There is a want for hardware optimised to perform improved non-invasive brain stimulation using ultrasound, or otherwise perform therapeutic and diagnostic ultrasound applications in the brain.

### SUMMARY

According to a first aspect, there is provided a transcranial ultrasound cap, the cap comprising:
a bowl having an inner volume arranged to receive a portion of a human head, the bowl conforming to the shape of an ellipsoid; and
a transducer array in the inner volume, comprising a plurality of ultrasound transducers coupled to the bowl.

The bowl may be the shape of a portion of an ellipsoid. For example, the bowl may have a substantially half-ellipsoidal shape. The cap may comprise one or more parts that attach to the bowl and that conform to the ellipsoid shape once attached—for example a seal, as described below.

The bowl may be another shape—for example, substantially hemispherical or substantially half-ovoidal. The bowl may be any suitable shape other than hemispherical, for example. The transducer array may be used with any suitably shaped bowl. Any of the features set out herein may be used with a bowl that does not conform to the shape of an ellipsoid.

According to a second aspect, there is provided a transcranial ultrasound cap, the cap comprising:
a bowl having an inner volume arranged to receive a portion of a human head; and
a transducer array in the inner volume, comprising a plurality of ultrasound transducers coupled to the bowl;
wherein the bowl comprises an exclusion zone in a central region thereof, and the ultrasound transducers are arranged outside of the exclusion zone.

According to a third aspect, there is provided a transcranial ultrasound cap, the cap comprising:
a bowl having an inner volume arranged to receive a portion of a human head;
a transducer array in the inner volume, comprising a plurality of ultrasound transducers coupled to the bowl; and
a base configured to couple the cap to a horizontal bed;
wherein the bowl comprises a front surface that defines an opening of the bowl and the front surface is inclined, relative to a plane that is perpendicular with the horizontal bed.

The optional features described below are applicable to any of the first, second and third aspects.

The bowl may be re-usable (i.e. not patient specific).

The bowl may comprise a flange having a sealing surface around the perimeter of the bowl. The cap may comprise a seal to contain a liquid in contact with the inner surface of the bowl and in contact with the subject's head. The seal may be configured to form a seal with both the sealing surface of the flange and to form a seal with the subject's head. The seal may comprise an outer sealing ring for forming a seal with the sealing surface of the flange. The seal may comprise an inner seal configured to form a seal with the subject. The inner seal may be patient specific (e.g. formed from a cast of the subject or by rapid prototyping). The seal may have a flat annular ring shape.

The central region may have no transducers, or a reduced density of transducers.

The bowl of the first or third aspect may comprise an exclusion zone in a central region of the bowl, wherein the ultrasound transducers are arranged outside of the exclusion zone, according to the second aspect. The bowl of the second or third aspect may conform to an ellipsoid.

The cap may be insertable into an MRI scanner or other scanning or imaging machine, and configured for use with the machine (i.e. free from ferromagnetic materials).

The transducers may be distributed on the bowl in a random array (or quasi-random array) with a minimum spacing of between 1 mm and 20 mm. The minimum spacing may be between 5 mm and 15 mm. In some examples, the minimum spacing may be 10 mm. The mean spacing may be at least 10 mm. The transducers may be arranged in a random array within a segment of the bowl, such that the bowl is more densely populated with transducers in that segment than in other parts of the bowl.

The transducers may have normal axes oriented towards a geometric centre of the bowl. In use, this may be where a target area of the wearer's head (which may be the deep brain) is positioned.

The transducers may be attached to the bowl in a push-fit arrangement, the cap further comprising a removable fastener associated with each transducer. The removable fastener may be arranged on a rear surface of the bowl outside of the inner volume. The transducers may be arranged in perforations in the bowl, with parts of the transducers adjacent the front surface and parts for coupling the transducers to the bowl adjacent the rear surface, for attachment to the removable fasteners.

Instead of a removable fastener associated with each transducer, a removable fastener may be used to couple multiple transducers to the bowl. However, this may be less suitable where the transducers are arranged in a random array as the separation between transducers on the bowl would be variable.

Each transducer may have a circular face arranged with the centre of the face positioned on the front surface of the bowl. This may create a smooth or substantially smooth inner surface (or the face of the transducer may protrude from the inner surface of the bowl). Where the transducer is inserted into a perforation in the bowl, the face may have a cross section substantially equal to a cross section of the perforation such that the transducer closes the perforation when inserted therein. Liquid may be introduced into the bowl and closing the perforation with the transducer may effectively prevent leaking. The cross section of the face may be smaller than the cross section of the perforation, to allow easy insertion of the transducer. Any gap between the face and the perforation may be filled with a filler—for example silicone grease—to create a watertight or substantially watertight seal.

The transducers may have circular or substantially circular cross-sections, and each may have an active element (for example, a piezoelectric element) with a diameter of less than 5 mm. For example, one or more transducers may have an active element diameter of 3 mm. Each may have an active element diameter of 3 mm. Use of such small transducers may increase the steering range and reduce grating lobes that may otherwise arise in a sparse random array.

The cap may further comprise an inlet configured to deliver a liquid into the inner volume of the bowl.

The cap may further comprise an outlet for removal of liquid from the inner volume of the bowl (so that liquid can be circulated via the inlet and outlet). The outlet may be arranged adjacent the opening to the inner volume. The outlet may be arranged generally opposite the inlet.

The bowl may define an exclusion zone, which may be free of transducers (or have a reduced density of transducers). The length and the width of the exclusion zone may each exceed the mean spacing between transducers—i.e. merely a space between two transducers may not constitute an exclusion zone.

The exclusion zone may be located in a central region of the bowl.

The cap may comprise an exclusion zone (with no transducers or a reduced density of transducers) including the inlet. The outlet may be positioned in an exclusion zone. An edge exclusion zone may be defined part way or all the way around the edge of the bowl and both the inlet and outlet may be arranged in the edge exclusion zone.

The cap may include more than one exclusion zone—for example, at the centre of the bowl and at the edge of the bowl. The central exclusion zone may allow elements to be connected to the bowl—for example, part of the inlet or outlet, part of an MRI scanner or part of an apparatus for positioning a subject. For example, a boss may be provided on the cap in an exclusion zone, for connecting the cap to a support structure.

An inner surface of the bowl may conform to a semi-ellipsoid, for example, it may have three principal semi-axis dimensions as follows: a length of 90 mm to 110 mm plus an offset distance, a width of 70 mm to 90 mm plus an offset distance, and a height of 85 mm to 105 mm plus an offset distance. The length may be 103 mm. The width may be 78 mm. The height may be 95 mm. The offset distance may be between 30 mm and 50 mm. For example, the offset distance may be 40 mm. The offset distance in combination with the specific dimensions have been found by the applicant to provide a substantially fixed offset from a typical person's head, which may be advantageous.

An offset angle may be defined from a plane of a geometric centre of the bowl and the transducers may be arranged in a random array within an area of the bowl above an offset angle of 10 degrees to 30 degrees, for example 15 degrees.

An exclusion zone may be included below the offset angle. For example, the exclusion zone may be included within an angle of 15 degrees of a plane of the opening to the inner volume.

The transducers may be arranged on the bowl within a segment angle above the offset angle, wherein the segment angle may be between 35 degrees and 65 degrees (or 45 to 60 degrees), for example wherein the segment angle is 55 degrees.

The transducer array may comprise between 128 and 1024 ultrasound transducers. For example, between 128-512 transducers. The transducer array may comprise 256 individual ultrasound transducers.

The cap may comprise a base configured to couple the cap to a horizontal bed. The bowl may comprise a front surface that defines an opening of the bowl and the front surface may be inclined, relative to a plane that is perpendicular with the horizontal bed.

The bowl may have dimensions to be worn on a human head at a bowl angle of 15 degrees to 40 degrees, wherein the bowl angle is defined as the angle between the plane of the front surface and an axial plane. The bowl may have dimensions to be worn on a human head at a bowl angle of 20 degrees, for example.

The bowl may be configured to accommodate an amount of human hair and/or an amount of liquid. The bowl may be dimensioned to have a clearance of a minimum of 25 mm between a head ellipse of an average adult head and the inner surface of the bowl. The bowl may have a clearance of 40 mm either side of the head ellipse of an average human adult head.

The cap may further include a fixture configured to attach the cap to a magnetic resonance imaging machine and/or to a subject positioning apparatus configured to position the subject for placing their head in the bowl.

Another aspect provides a transcranial ultrasound device comprising the cap, the cap comprising an inlet and an outlet, the cap further comprising a circulator configured to pass water into the inner volume via the inlet and out of the inner volume via the outlet. The circulator may further comprise a conditioner configured to alter the composition or temperature of the liquid in the circulator. The circulator may be configured to pass liquid from the outlet to the inlet, for reuse.

The transcranial ultrasound device may further comprise a subject positioning apparatus to position a subject with respect to the cap.

Another aspect provides a system, comprising: the cap according to the first, second or third aspects (including any optional features disclosed herein), and a subject positioning apparatus. The subject positioning apparatus may be configured to position the subject relative to the cap. The subject positioning apparatus may be patient specific and configured to conform with a subject's anatomy (e.g. in contact with the patient, or with a clearance of less than 2 mm). The subject positioning apparatus may comprise a face part and a neck part that are configured to fix together about the subject. The subject positioning apparatus may comprise a flange that is configured to clamp to the cap. The cap seal may be configured to be clamped between the flange of the cap and the flange of the subject positioning apparatus.

The features of each aspect (including optional features) may be combined with those of any other aspect.

### BRIEF DESCRIPTION OF THE DRAWINGS

Example embodiments will be described, by way of example only, with reference to the drawings, in which:

FIG. 1 shows a front elevation of a cap according to examples;

FIG. 2 shows a depiction of a bowl on a human head and labels planes and sizes of the bowl;

FIG. 3 shows a rear elevation of the cap according to examples;

FIG. 4 shows an exploded view of a transducer according to examples;

FIG. 5 shows a cross section of the cap on a human head according to examples;

FIG. 6 shows a model of the transducer positions on the inner bowl surface according to examples;

FIG. 7 shows an exploded view of the cap, a seal and a subject positioning apparatus according to examples; and

FIG. 8 shows the cap according to examples.

### DETAILED DESCRIPTION

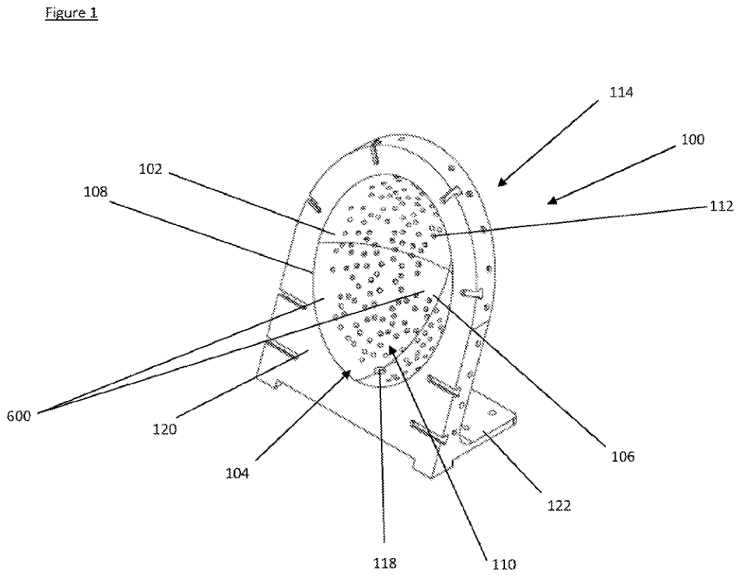

FIG. 1 shows an example embodiment, comprising a cap 100 for use in TUS. The cap 100 may be configured to deliver targeted low-intensity ultrasound waves into the deep brain of a human (for example, an adult human). The cap 100 may be operable to modulate brain activity. Low intensity means less than 100 W per square centimeter spatial-peak pulse-average intensity at a focal position.

The cap 100 may be configured for use with software that controls the cap 100. The software may run a treatment plan and control the cap 100 based on the treatment plan.

The cap 100 comprises a bowl 102 with an inner volume 104 adjacent the inner surface 106. The inner surface 106 is curved and has an edge forming an opening 108 of the bowl 102.

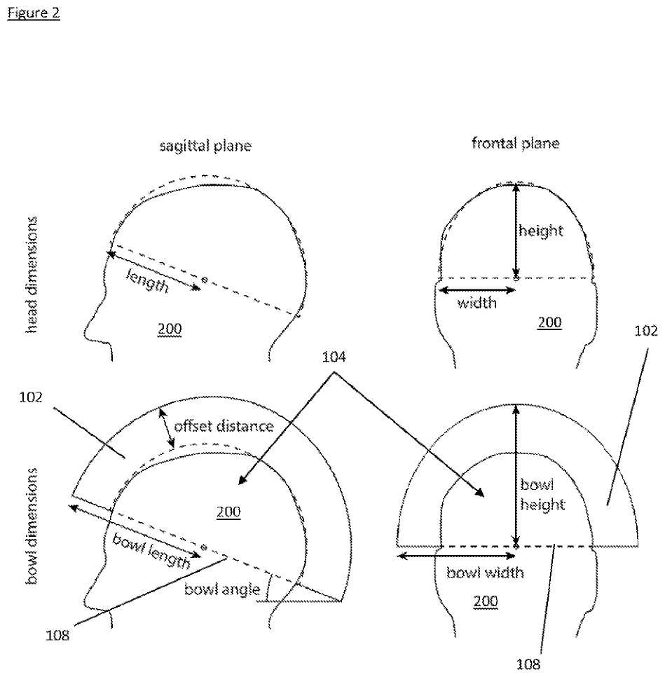

The cap 100 is arranged to be worn on a human head, as shown in FIG. 2. The bowl 102 may be dimensioned to receive a human head into the inner volume 104. For ease of description, any placement of the head into the bowl 102 is described as ‘wearing’ the cap 100. For practical reasons, the subject to be treated using the cap 100 would insert their head into the bowl 102 from a supine position (e.g. lying on the bed of an MRI). The cap 100 may be fixed to a horizontal bed (e.g. of an MRI) as part of a wider system. The cap 100 may be completely non-invasive, both in terms of hardware and any treatment applied using the hardware.

The bowl 102 may have a substantially half-ellipsoidal shape, as shown in FIG. 1. The substantially half-ellipsoidal shape may conform with the shape of a typical person's head better than a hemispherical shaped bowl, for example. This improved conformity with the head may have a number of advantages. For a specific minimum clearance, the average distance from the bowl to the subject's head will be minimised by an ellipsoidal shape. This reduces the amount of power required at the transducers to achieve a specific level of acoustic intensity within the brain. The volume of water around the head is similarly minimised, which may be advantageous for MRI imaging of subjects within the bowl. The reduced volume of water may reduce dielectric artefacts and the field-of-view needed to avoid wrap around aliasing artefacts.

It is important that the subject's head is held still with respect to the bowl 102 (or vice versa, for example where the cap 100 is not in a fixed position), so that the correct region of their brain may be stimulated. A subject positioning apparatus may be attached to the cap 100 to accurately position the subject within the cap 100.

The shape of the bowl 102 may be selected based on the average shape of a human adult head, so that the bowl 102 conforms well to the subject. The bowl 102 may have dimensions conformal to an average adult human head. The lengths of the semi-axes of the half-ellipsoid may conform to an average adult human head.

A plane of the opening 108 to the inner volume 104 may be coincident or substantially coincident with a planar cross-section of the skin surface surrounding the neurocranium having the greatest area, when worn, as shown in FIG. 2. A seal 900 at this plane may be more effective.

The cap 100 comprises a transducer array 110, arranged in the bowl 102. The transducer array 110 may be provided for stimulation of the wearer's brain, using ultrasound.

The bowl 102 may comprise perforations 406 and ultrasound transducers 112 may be received in each perforation 406. The plurality of ultrasonic transducers are configured to be driven as a phased array, so that ultrasound can be focused on targeted regions of the brain, for example in accordance with a treatment plan (which may be determined in dependence on the results from scanning, for example by MRI). The recesses/perforations 406 may be spread out over the bowl 102 surface.

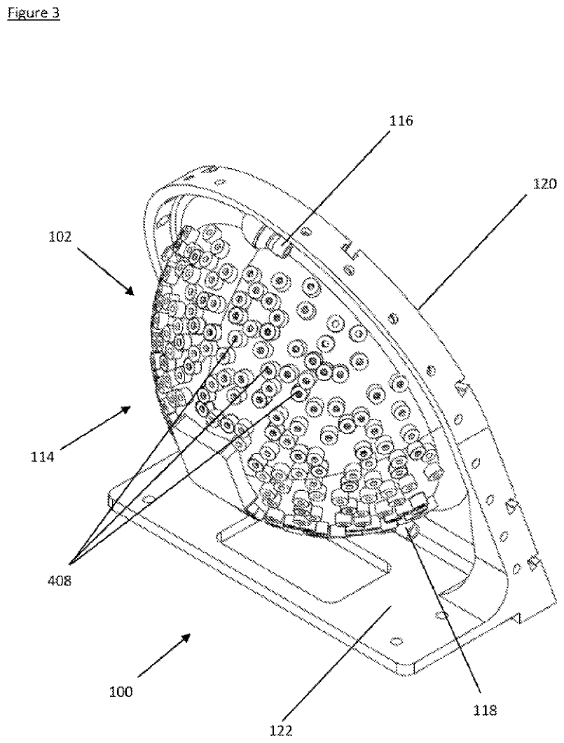

A rear view of the bowl 102 is shown in FIG. 3, in which the rear surface 114 of the bowl 102 is visible.

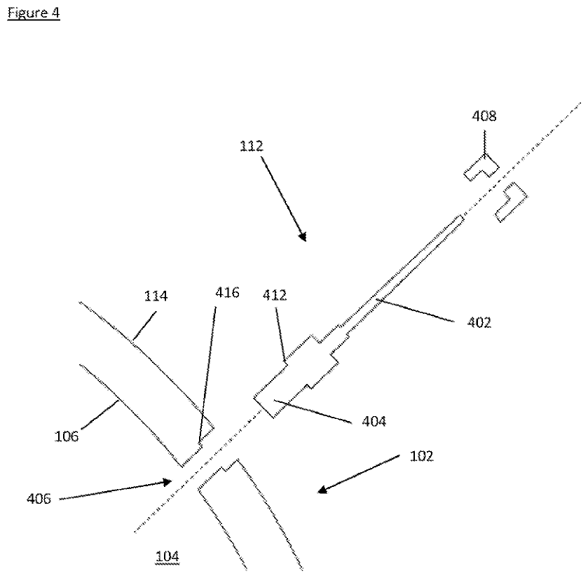

FIG. 4 shows how an (or each) ultrasound transducer 112 may be coupled to the bowl 102.

The example transducer 112 comprises a tip portion 404, and transducer flange 412 and connector for connection to cable 402. The perforation 406 comprises a stepped hole, comprising a first portion adjacent the inner surface 106 with a first diameter, and a second portion adjacent the outer surface 114 with a second larger diameter. A shoulder 416 separates the first and second portion of the perforation.

The shoulder 416 engages with the transducer flange 412 to accurately position the transducer 112 in the perforation, with the tip portion 404 received in the first portion of the perforation 406. The tip portion 404 may subsequently be positioned with an outer face substantially coincident with the inner surface 106.

Each transducer 112 may point towards a common position, for example the geometric centre of the bowl surface, corresponding with a position deep within the subject's brain. For this reason, the surface of the tip portion 404 may not exactly conform with the bowl surface. Furthermore, the front surface of the tip portion 404 may be flat—it will be appreciated that a flat front surface of the tip portion will not exactly conform to a curved bowl surface. The centroid of the front surface of the tip portion may be configured to coincide with the inner surface of the bowl. In other embodiments, the centroid of the front surface of the tip portion may be configured to protrude a fixed distance past the inner surface of the bowl (for example, by less than 5 mm). This offset ensures that the front surfaces of all transducers are clear of the perforations which avoids any acoustic shadowing effects that might occur if the front surfaces were partially or wholly within the perforations. The dimensions of the bowl surface may be adjusted to compensate for this offset. In an example case where the offset is 2 mm, the inner bowl surface may be enlarged by 2 mm to compensate (so that the centroid of the transducers are in the desired conformation, and the perforations cannot shadow the transducers).

Silicon grease may be used to assist in forming a seal between the transducer 112 and the bowl 102.

The connector may comprise a removable (e.g. without tools, for example push fit, screw fit etc) electrical connector, so that electrical connections to transducer elements may readily be reconfigured (e.g. in accordance with the requirements of a particular treatment plan). For example, where there are more transducers than electrical elements, this may enable the transducers that are “live” to be reconfigured.

The perforation 406 and a cross section of the transducer tip portion 404 may be circular.

The tip portion 404 may contain an active element (for example, a piezoelectric element) with a diameter of less than 5 mm—for example, 3 mm. The small transducer size allows many transducer elements 112 to be employed and may allow an improved electronic steering range versus caps that use larger elements. The cap 100 may be able to steer a beam formed by the transducer elements 112 at least 50 mm from the geometric focus 500 of the bowl 102 (by adjustment of the relative phase and amplitude of signals applied to different transducers).

A removable fastener in the form of transducer cap 408 may be glued semi-permanently to the bowl 102 to retain the transducer 112 in the perforation 406.

Each of the elements shown in FIG. 4 may be non-ferrous, to avoid missile hazards during MRI scanning.

Ultrasound transducers 112 may be removed and replaced, and may be inserted into a different perforation 406 if desired. The cap 100 may therefore be easily reconfigurable and it may be simple to repair (by removing only non-functioning ultrasound transducers 112). In some embodiments, some perforations 406 may be filled with plugs (rather than transducers 112). The perforations 406 that are filled with transducers 112 may be selected based on a treatment plan.

As an alternative to securing or fastening the transducer 112 with the transducer cap 408 using adhesive, the transducers 112 (or transducer caps 408) may comprise a threaded portion, and the perforation 406 may be threaded. In some embodiments, there may be a secure interference fit between the transducer 112 and the bowl 102.

The ultrasound transducers 112 may be distributed on the bowl 102 in a random array. This may have the effect of minimizing grating lobes.

There may be a minimum spacing assigned to the random array (such that the array may be considered to be pseudo-random, in the sense that there is a minimum spacing, but that above the minimum spacing placement of ultrasound transducers 112 is random). The minimum spacing may be between 1 mm and 20 mm, for example. The minimum spacing may be between 5 mm and 15 mm. The minimum spacing may be 10 mm. In some embodiments, positions may be selected based on a probability distribution function that has bounds on the minimum and/or maximum spacing. In practice, this results in the spacing between different transducers 112 being generally different.

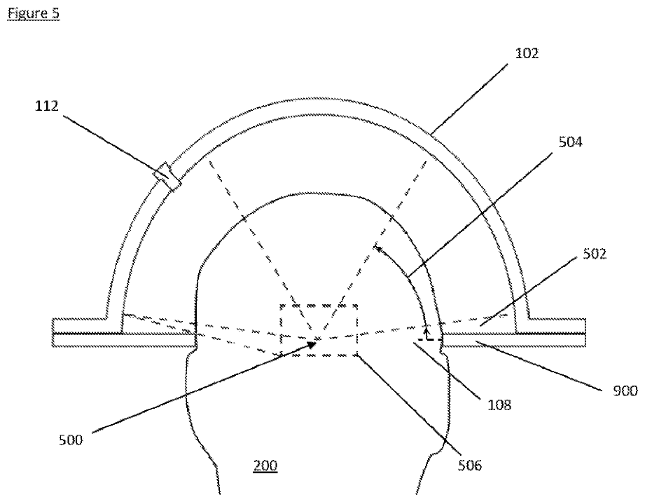

An offset angle 502 may be defined from the bowl centre 500—as shown in FIG. 5. The offset angle 502 may be measured from a plane of the opening 108. The offset angle 502 may define an edge exclusion zone 600 of the bowl 102 which does not contain any ultrasound transducers 112. The offset angle 502 may be between 10 and 30 degrees. The offset angle 502 may be less than 20 degrees, e.g. 15 degrees.

The ultrasound transducers 112 may be arranged in a random array within an area of the bowl 102 above the offset angle 502.

The bowl 102 may be attached to a seal 900 (as described below) at its edge, and so positioning the transducers 112 above the offset angle 502 allows all transducers 112 to have a line-of-sight to the deepest target within the brain. The plane of the opening 108 from which the offset angle 502 is defined may be a plane of the opening 108 in line with the seal 900 as shown in FIG. 5. FIG. 5 shows a steering range box 506 around the bowl centre 500 over which a focal point from the array can be steered.

The corner of the box is limited by line of sight with the inner surface of the bowl over the edge of the seal 900. The seal 900 may be relatively thin (normal to the plane of the seal) thereby enabling the extent of the steering range box 506 to be maximised. The thickness of the seal 900 may be less than 20 mm, or less than 15 mm.

The transducers 112 may each have a normal axis directed to the geometric centre 500 of the bowl 102 in order to reach the deep brain at 506.

A central part of the bowl 102 (opposite the opening 108) may also be free of ultrasound transducers 112; this may be an exclusion zone 600.

The random array of ultrasound transducers 112 may be arranged within a segment angle 504 of the bowl 102 defined above the offset angle 502 towards a peak of the bowl 102 (opposite the opening 108).

The ultrasound transducers 112 may be arranged on the bowl 102 within a segment angle 504 of between 35 degrees and 65 degrees above the offset angle 502. For example, the transducers 112 may be arranged on the bowl 102 within an angle of 55 degrees above the offset angle 502. The size of the offset angle 502 may determine how large the segment angle 504 of the bowl 102 including the ultrasound transducers 112 can be—if the maximum possible total of offset angle 502 plus segment angle 504 is 90 degrees, and it is desired to keep part of the bowl 102 opposite the opening 108 clear of transducers 112, then the total of offset angle 502 plus segment angle 504 will be less than 90 degrees.

Not populating the central region of the bowl 102 with transducers 112 (i.e. providing an exclusion zone 600 above the segment angle 504) has the effect of increasing transducer density at side of the bowl 102. The offset angle 502 allows line of sight between all the transducers 112 and a target volume. This combination of factors improves acoustic performance.

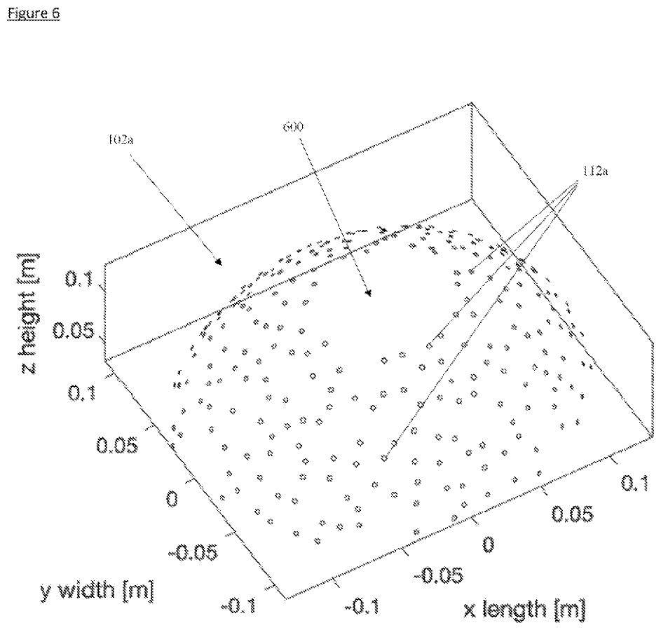

A regular array of elements with spacing greater than half the acoustic wavelength may lead to significant grating lobes. However, arranging the transducers 112 in a random array addresses this potential problem. A set of positions for the transducers 112 within the segment angle 504 and above the offset angle 502 may be determined as the set which gives the minimum product of side lobe height and focal size. Other positions may work but may not be optimal. An example of a well optimised set of positions is shown in FIG. 6. FIG. 6 models the bowl 102 and transducers 112 (marked as 102a and 112a on the model, for clarity).

The cap 100 may comprise an exclusion zone 600—an area on the surface (front and/or rear) 106, 114 free of transducers 112. The spacing between the ultrasound transducers 112 around the outside of the exclusion zone 600 may be such that there is sufficient space to attach something to the bowl 102 without affecting the transducer array 110 (for example, without covering a transducer 112). A liquid connection may be made with the bowl 102 in an exclusion zone 600, for example—an input and output are described below.

Outside of the exclusion zone 600, there is an increased transducer density, in order to reduce grating lobes. For a given number of transducers 112, a greater transducer density may be achieved by using an exclusion zone 600 in the central region of the bowl 102.

The exclusion zone 600 may have an area of at least 10 mm² or at least 20 mm² for example. The exclusion zone 600 can be seen in the model of FIG. 6 as the region without any transducers 112a.

In an embodiment, sixteen healthy adult volunteers were observed in order to determine example dimensions for a bowl 102. The subjects lay in an MRI scanner in a comfortable position, and their head angle in the sagittal plane was used to calculate an optimal bowl angle of the transducer array 110 relative to a couch/bed of the MRI scanner. The average head size was then obtained by fitting a semi-ellipsoid to segmented structural MRI images.

The bowl 102 may have dimensions to be worn on a human head 200 at a bowl angle of 15 degrees to 40 degrees, wherein the bowl angle is defined as the angle between the plane of the front surface and an axial plane (as shown in FIG. 2). The bowl 102 may have dimensions to be worn on a human head 200 at a bowl angle of 20 degrees.

The dimensions of the bowl 102 were considered for rotation angles of 15 to 25 degrees—5 degrees each side of optimal—to account for small movements of the head.

The average head ellipse length (of an ellipse fitted to the largest area slice as shown in FIG. 2) was computed as 103 mm. The average head ellipse width (of an ellipse fitted to the largest area slice as shown in FIG. 2) was computed as 78 mm. The average head height was computed as 95 mm. The length:width:height ratio of the ellipsoid semi-axes was found to be 1.3:1.0:1.2 (to two significant figures).

The inner surface of the bowl 102 must be offset from the average dimensions, to allow for variations in anatomy. For example, an offset of 40 mm may be added to the principle semi-axis dimensions.

The offset may be chosen to allow keeping a sufficient volume of liquid (for example, water) around the wearer's head in use, as well as allowing room for hair where head shaving is not performed, and minimising the distance as much as possible to reduce required power for a given target intensity.

A different offset may be chosen—for example between 30 mm and 50 mm, or a larger or smaller distance as appropriate.

Including a 40 mm offset, a semi-ellipsoidal bowl 102 with principle semi-axis lengths of 143 mm (length), 118 mm (width), and 135 mm (height) was selected for the example embodiment.

The cap 100 may comprise an inlet 118 configured to deliver a liquid into the inner volume 104 of the bowl 102 as described below. The inlet 118 is in fluid communication with the inner volume 104. The inlet 118 includes an aperture through which liquid can be introduced into the inner volume 104. A chamber may be provided for holding liquid to be introduced into the inner volume 104 (e.g. by a pipe connecting the inlet to the chamber). A circulator and temperature regulator may be provided to control circulation and the temperature of the water (e.g. holding it at body temperature).

The inlet 118 may be arranged at the bottom of the inner volume 104 (near the rim at the bottom of the bowl 102) when the cap 100 is in use, so that the bowl 102 can be filled without leaving voids (e.g. from the bottom upwards). An outlet 116 may be provided at the top of the inner volume 104 when the cap 100 is in use (i.e. near the rim at the top of the bowl 102).

When the wearer is wearing the cap 100, the liquid from the inlet 118 may be received in the inner volume 104 of the bowl 102 in a volume defined between the bowl 102 and the wearer's head.

Introduction of a liquid (for example, water) into the cap 100 may facilitate acoustic coupling of the ultrasound transducers 112. The liquid may be any suitable ultrasonic transmission medium that is sufficiently well matched in ultrasonic transmission properties to the subject. Water is a convenient and suitable ultrasonic transmission medium.

As already discussed, the bowl 102 conforming to an ellipsoid, for example having a substantially half-ellipsoidal shape, means that the distance between each transducer 112 and the wearer's head, in use, is approximately equal. This has the benefit of minimising the effects of any sound speed variations due to temperature gradients in the liquid between the bowl 102 and the head. The waves from all of the transducers 112 will travel through approximately the same amount of liquid.

The cap 100 may comprise a flange comprising a sealing surface 120 around the perimeter of the bowl 102. The flange may include one or more slots for fixing the cap 100 to a subject positioning apparatus 908, as described below. The cap 100 may further comprise a base 122 extending in the superior direction away from the rear surface 114 of the bowl 102. The base 122 may be configured to be fixed to a horizontal bed of a scanning machine, for example.

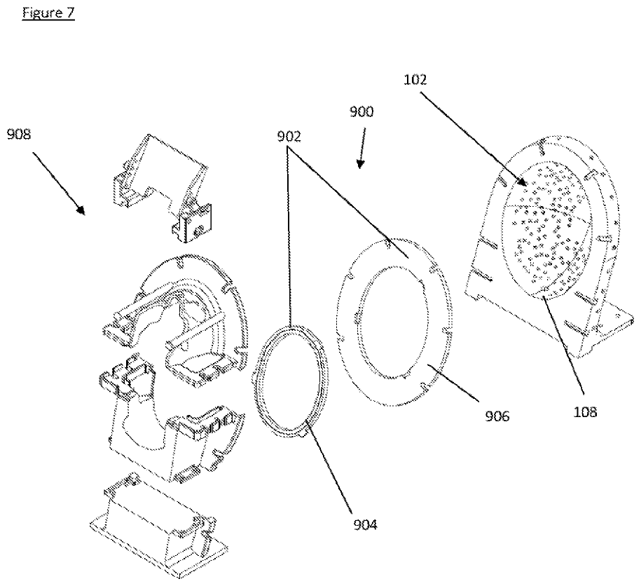

The cap 100 may include a seal 900, to prevent liquid leaking out of the inner volume 104 (from anywhere other than the outlet 116). FIG. 7 shows an exploded view of the cap 100, seal 900 and subject positioning apparatus 908. The seal 900 may comprise outer sealing ring 906 and inner seal 904. The inner seal 904 is configured to form a seal with the subject, and the outer sealing ring is configured to form a seal with the sealing surface 120 of the cap 100.

The seal 900 may also facilitate stabilising the cap 100 with respect to the wearer's head 200, since the inner seal 904 is in contact with the subject's head 200 in use. The inner seal 904 may include a compressible portion that is configured to contact the head 200.

The outer seal ring 906 may comprise a relatively rigid material. A sealing element (not shown) may be provided between the outer seal ring 906 and the sealing surface 120 of the cap 100.

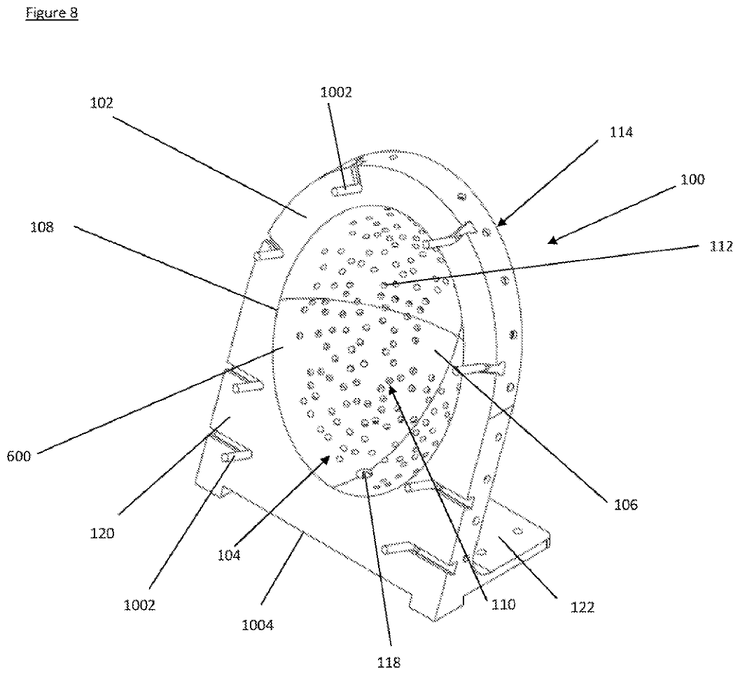

The cap 100 may comprise at least one fastener 1002 for attaching the cap 100 to the subject positioning apparatus 908, as shown in FIG. 8.

The cap 100 may be used alongside a mask or neck rest, for example, or another frame to keep the wearer's head still at a predefined position. The cap 100 may be attached to a couch on which the wearer can lie to keep still and to maintain a desired angle of the head (and/or neck) with respect to the cap 100. The cap 100 may comprise fixtures and fasteners for attaching the cap 100 to a mask, neck rest, couch or other frame (collectively referred to here as ‘subject positioning apparatus’ 908, and including any equipment that may be used to position the wearer ready to have their head received in the bowl 102 of the cap 100).

The fixtures 1002 may pass through the seal 900 to attach the cap 100 to other elements of the subject positioning apparatus 908, or separate fixtures 1002 may be provided accordingly.

The fastener 1002 may comprise a bolt or screw and the cap 100 may define one or more channels configured to receive the bolt or screw or other suitable fixing piece.

The fastener 1002 may be formed from a non-ferrous material. The fastener 1002 may comprise a hexagonal bolt and a wingnut, for example.

The fixture 1002 may be a quick-release fixture 1002. As the wearer may be encased in the cap 100 and a face mask, for example, it may be desirable to offer a quick means of removing anything holding the wearer in place.

The subject positioning apparatus may first be placed on the subject, then the subject positioned adjacent the cap 100, then the subject positioning apparatus 908 secured to the cap 100.

The cap 100 may include a slot 1004 to receive a part of the subject positioning apparatus 908, for example, to slidably receive a part (e.g. base) of the subject positioning apparatus 908.

The subject positioning apparatus 908 may be customized (for example 3D printed) to conform with a subject's anatomy for positioning a particular subject with respect to the cap 100.

Although the appended claims are directed to particular combinations of features, it should be understood that the scope of the disclosure of the present invention also includes any novel feature or any novel combination of features disclosed herein either explicitly or implicitly or any generalisation thereof, whether or not it relates to the same invention as presently claimed in any claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable sub combination.

The examples provided in the detailed description are intended to provide examples of the invention, not to limit its scope, which should be determined with reference to the accompanying claims.

What is claimed is:

1.-23. (canceled)

24. A transcranial ultrasound cap, the cap comprising:
a bowl having an inner volume arranged to receive a portion of a human head, the bowl conforming to the shape of an ellipsoid; and
a transducer array in the inner volume, comprising a plurality of ultrasound transducers coupled to the bowl.

25. The transcranial ultrasound cap of claim 24, wherein the bowl comprises an exclusion zone in a central region thereof, and the ultrasound transducers are arranged outside of the exclusion zone.

26. The transcranial ultrasound cap of claim 24, the cap further comprising:
a base configured to couple the cap to a horizontal bed;
wherein the bowl is configured with a front surface that defines an opening of the bowl and the front surface is inclined, relative to a plane that is perpendicular with the horizontal bed.

27. The cap according to claim 24, wherein the transducers are distributed on the bowl in a random array with a minimum spacing of between 1 mm and 20 mm.

28. The cap according to claim 24, wherein the transducers have normal axes oriented towards a geometric centre of the bowl.

29. The cap according claim 24, wherein the transducers are attached to the bowl in a push-fit arrangement, the cap further comprising a removable fastener associated with each transducer.

30. The cap according to claim 24, wherein the transducers have circular or substantially circular cross-sections, and each has an active element diameter of less than 5 mm.

31. The cap according to claim 24, further comprising an inlet configured to deliver a liquid into the inner volume of the bowl, the inlet arranged adjacent the opening to the inner volume; and/or an outlet for removal of liquid from the inner volume of the bowl, the outlet arranged adjacent the opening to the inner volume.

32. The cap according to claim 24, further comprising an edge exclusion zone adjacent an edge of the bowl, wherein the transducers are arranged outside of the exclusion zone.

33. The cap according to claim 24, wherein an inner surface of the bowl has a substantially semi-ellipsoidal shape with principal semi-axis lengths of 90 mm to 110 mm plus an offset distance (length), 70 mm to 90 mm plus an offset distance (width) and 85 mm to 105 mm an offset distance (height).

34. The cap of claim 33, wherein the offset distance is between 30 mm and 50 mm.

35. The cap according to claim 24, wherein an offset angle is defined from a plane of the geometric centre of the bowl and the transducers are arranged in a random array within an area of the bowl above an offset angle of between 10 and 30 degrees.

36. The cap according to claim 35, wherein the transducers are arranged on the bowl within a segment angle above the offset angle, wherein the segment angle is between 35 degrees and 65 degrees.

37. The cap according to claim 24, wherein the transducer array comprises between 128 and 1024 ultrasound transducers or wherein the transducer array comprises 256 individual ultrasound transducers.

38. The cap according to claim 24, wherein a plane of the opening to the inner volume is coincident or substantially coincident with a planar cross-section of the human head having the greatest area, when worn.

39. The cap according to claim 24, wherein the bowl has dimensions to be worn on a human head at a bowl angle of 15 degrees to 40 degrees, wherein the bowl angle is defined as the angle between the plane of the front surface and an axial plane.

40. The cap according claim 24, wherein the bowl has dimensions to be worn on a human head at a bowl angle of 20 degrees, wherein the bowl angle is defined as the angle between the plane of the front surface and an axial plane.

41. The cap according to claim 24, further including a fixture configured to attach the cap to a magnetic resonance imaging machine and/or to a subject positioning apparatus configured to position the subject for placing their head in the bowl.

42. A transcranial ultrasound device comprising a transcranial ultrasound cap, the transcranial ultrasound cap comprising:
a bowl having an inner volume arranged to receive a portion of a human head, the bowl conforming to the shape of an ellipsoid;
a transducer array in the inner volume, comprising a plurality of ultrasound transducers coupled to the bowl;
where the cap comprises an inlet and an outlet, and
the device further comprising a circulator configured to pass water into the inner volume via the inlet and out of the inner volume via the outlet.

43. The transcranial ultrasound device of claim 42, further comprising a subject positioning apparatus to position a subject with respect to the cap.
